SEQUENCE LISTING (2 sequences)

SEQ ID NO: 1 - protein, 210 aa, Homo sapiens
MADGSSDAAREPRPAPAPIRRRSSNYRAYATEPHAKKKSKISASRKLQLKTLLLQIAKQELEREAEERRGEKGRALSTRCQPLELAGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

SEQ ID NO: 2 - DNA, 843 bp, Homo sapiens
agtgtcctcggggagtctcaagcagcccggaggagactgacggtccctgggaccctgaaggtcacccgggcggccccctcactgaccctccaaacgcccctgtcctcgccctgcctcctgccattcccggcctgagtctcagcatggcggatgggagcagcgatgcggctagggaacctcgccctgcaccagccccaatcagacgccgctcctccaactaccgcgcttatgccacggagccgcacgccaagaaaaaatctaagatctccgcctcgagaaaattgcagctgaagactctgctgctgcagattgcaaagcaagagctggagcgagaggcggaggagcggcgcggagagaaggggcgcgctctgagcacccgctgccagccgctggagttggccgggctgggcttcgcggagctgcaggacttgtgccgacagctccacgcccgtgtggacaaggtggatgaagagagatacgacatagaggcaaaagtcaccaagaacatcacggagattgcagatctgactcagaagatctttgaccttcgaggcaagtttaagcggcccaccctgcggagagtgaggatctctgcagatgccatgatgcaggcgctgctgggggcccgggctaaggagtccctggacctgcgggcccacctcaagcaggtgaagaaggaggacaccgagaaggaaaaccgggaggtgggagactggcgcaagaacatcgatgcactgagtggaatggagggccgcaagaaaaagtttgagagctgagccttcctgcctactgcccctgccctgaggagggccctgaggaataaagcttctctctgagctgaaa

(19) United States
(12) Patent Application Publication
(10) Pub. No.: US 2022/0184232 A1
(43) Pub. Date: Jun. 16, 2022

(54) METHODS OF TREATING TNNI3-MEDIATED CARDIOMYOPATHY

(71) Applicant: The Regents of the University of California, Oakland, CA (US)

(72) Inventors: Eric Adler, La Jolla, CA (US); Paul Bushway, La Jolla, CA (US)

(21) Appl. No.: 17/440,099

(22) PCT Filed: Mar. 24, 2020
(86) PCT No.: PCT/US2020/024477

§ 371 Date: Sep. 16, 2021

Related U.S. Application Data
(60) Provisional application No. 62/823,467, filed on Mar. 25, 2019.

(51) Int. Cl. A61K 48/00; C12N 15/86

(52) U.S. Cl. CPC A61K 48/0058; C12N 2750/14143; C12N 15/86

(57) ABSTRACT

Provided herein are gene therapy vectors containing a polynucleotide encoding a cardiac-specific promoter operably linked to a polynucleotide encoding Troponin I3, and methods of using such vectors for preventing, mitigating, ameliorating, reducing, inhibiting, eliminating and/or reversing one or more symptoms of cardiomyopathy.

### CROSS REFERENCE TO RELATED APPLICATION(S)

This application claims the benefit of priority under 35 U.S.C. § 119(e) of U.S. Ser. No. 62/823,467, filed Mar. 25, 2019, the entire content of which is incorporated herein by reference.

### SEQUENCE LISTING

The instant application contains a Sequence Listing which has been submitted electronically in ASCII format and is hereby incorporated by reference in its entirety. Said ASCII copy, created on Mar. 24, 2020, is named 20378-202473_SL.txt and is 4 kilobytes in size.

### BACKGROUND OF THE INVENTION

### Field of the Invention

The invention relates generally to cardiomyopathy and more specifically to treatment of such diseases with gene therapy.

### Background Information

Cardiomyopathy is a disease of the heart muscle that makes it harder for the heart to pump blood to the rest of the body. If left untreated, cardiomyopathy can lead to heart failure. The main types of cardiomyopathy include dilated, hypertrophic and restrictive cardiomyopathy. As the condition progresses, the typical signs and symptoms of cardiomyopathy include, but are not limited to, breathlessness with or without exertion; swelling of legs, ankles, and feet; bloating of the abdomen due to fluid buildup; coughing while lying down; fatigue; rapid, pounding of fluttering heartbeats; chest discomfort or pressure; and dizziness, lightheadedness and fainting. Current treatment includes medications, surgically implanted devices or, in severe cases, a heart transplant. However, current treatments are effective at slowing the rate of progression of the disease, but often fail to treat or prevent it. Thus, there is a need in the art for alternative or improved methods for treating and preventing cardiomyopathy.

### SUMMARY OF THE INVENTION

The present invention is based, in part, on the finding that an expression vector containing a nucleic acid molecule encoding human TNNI3 and a cardiac-specific promoter can be used to deliver TNNI3 to cardiomyocytes of the mammalian heart. Accordingly, in one aspect, the invention provides gene therapy vectors, e.g., for use in systemically or locally increasing the expression of Troponin I3 (TNNI3) in a subject. The gene therapy vectors find use in preventing, mitigating, ameliorating, reducing, inhibiting, and/or treating one or more symptoms of cardiomyopathy, heart failure or arrhythmia. In various embodiments, the gene therapy vectors include an expression cassette that includes a polynucleotide encoding a cardiac-specific promoter operably linked to a polynucleotide encoding a functional human TNNI3 gene. In various embodiments, the vector is a viral vector. In various embodiments, the viral vector is from a virus selected from the group consisting of adenovirus, retrovirus, lentivirus, herpesvirus and adeno-associated virus (AAV). In various embodiments, the vector is from one or more of adeno-associated virus (AAV) serotypes 1-11, or any subgroups thereof. In various embodiments, the viral vector is encapsulated in an anionic liposome. In various embodiments, the vector is a non-viral vector. In various embodiments, the non-viral vector is selected from the group consisting of naked DNA, a cationic liposome complex, a cationic polymer complex, a cationic liposome-polymer complex, and an exosome. In various embodiments, the expression cassette comprises operably linked in the 5′ to 3′ direction (from the perspective of the mRNA to be transcribed), a first inverse terminal repeat, an enhancer fused to the cardiac-specific promoter, the polynucleotide encoding TNNI3, a posttranscriptional regulatory element, and a second inverse terminal repeat. In various embodiments, the expression cassette also includes a ubiquitous chromatin open element positioned upstream of the enhancer. In various embodiments, the promoter is selected from the group consisting of TNNT2 proximal promoter and C5C12 synthetic promoter. In various embodiments, the enhancer is a 2RS5 muscle enhancer. In various embodiments, the polynucleotide comprises DNA or cDNA. In various embodiments, the polynucleotide encoding TNNI3 has at least about 90% sequence identity, e.g., at least about 91%, 92%, 93%, 94%, 95%, 96%, 97%, 98%, 99% sequence identity, to SEQ ID NO: 2. In various embodiments, the polynucleotide encoding TNNI3 is SEQ ID NO: 2.

In another aspect, provided are methods of preventing, mitigating, ameliorating, reducing, inhibiting, eliminating and/or reversing one or more symptoms of cardiomyopathy or arrhythmia in a subject in need thereof. The methods include administering to the subject a gene therapy vector as described above and herein.

In yet another aspect, provided are methods of preventing, mitigating, ameliorating, reducing, inhibiting, eliminating and/or reversing one or more symptoms of cardiomyopathy or arrhythmia in a subject in need thereof. The methods include administering to the subject an adeno-associated virus (AAV) vector comprising an expression cassette comprising a polynucleotide encoding TNNI3. In various embodiments, the expression cassette also includes a cardiac-specific promoter operably linked to the polynucleotide encoding TNNI3. In various embodiments, the expression cassette comprises operably linked in the 5′ to 3′ direction, a first inverse terminal repeat, an enhancer fused to the cardiac-specific promoter, the polynucleotide encoding TNNI3, a posttranscriptional regulatory element, and a second inverse terminal repeat. In various embodiments, the expression cassette also includes a ubiquitous chromatin open element positioned upstream of the enhancer. In various embodiments, the subject is a mammal, such as a human. In various embodiments, the vector is administered via a route selected from the group consisting of intravenous, intraarterial, intracardiac, intracoronary, intramyocardial, intrarenal, intraurethral, epidural, intracranial, subcutaneous, and intramuscular. In various embodiments, the vector is delivered or administered via a physical or mechanical method selected from the group consisting of microinjection, jet injection, particle bombardment, hydrodynamic infusion, electroporation, sonoporation, laser irradiation, magnetofection. In various embodiments, the vector is administered multiple times with or without immunosuppression or plasmapheresis of the patient. In various embodiments, the arrhythmia is selected from the group consisting tachcardia and bradycardia. In various embodiments, the subject has been identified as having a mutated TNNI3 gene. In various embodiments, the subject has been identified as having progressive cardiomyopathy or end-stage heart failure.

### BRIEF DESCRIPTION OF THE DRAWINGS

FIGS. 1A-1D are pictorial diagrams showing exemplary schematics of Adeno-Associated Virus LAMP-2 Gene Delivery Constructs. FIG. 1A shows an exemplary AAV vector coding for the 2RS5 muscle enhancer fused to the TNNT2 proximal promoter. FIG. 1B shows an exemplary AAV vector coding for a ubiquitous chromatin opening element (UCOE) derived from the CBX3 gene promoter. The UCOE provides approximately 3500 bp of protection from methylation-based gene silencing in the direction of the TNNI3 coding sequence. The UCOE is positioned upstream of the 2RS5 muscle enhancer fused to the TNNT2 proximal promoter. FIG. 1C shows an exemplary AAV vector coding for a UCOE positioned upstream of the 2RS5 muscle enhancer fused to a synthetic cardiac promoter C5C12. FIG. 1D shows an exemplary AAV vector coding for the 2RS5 muscle enhancer fused to a synthetic cardiac promoter (C5C12).

FIG. 2 is a pictorial diagram showing the results from western blots demonstrating that AAV9 is infectious in iPSC derived cardiomyocytes.

FIG. 3 is a pictorial diagram showing the results from western blots demonstrating the level and specificity of TNNI3 gene expression in mouse models receiving the gene therapy vectors.

### DETAILED DESCRIPTION OF THE INVENTION

The present invention is based, in part, on the finding that an expression vector containing a nucleic acid molecule encoding human TNNI3 and a cardiac-specific promoter can be used to deliver TNNI3 to cardiomyocytes of the mammalian heart.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “arrhythmia” refers to an improper beating of the heart, whether irregular, too fast, or too slow. Heart rhythm is normally controlled by a natural pacemaker (sinus node) located in the right atrium. The sinus node produces electrical impulses that normally start each heartbeat. These impulses cause the atria muscles to contract and pump blood into the ventricles. Arrhythmias are typically classified by the speed of the heart rate they cause, along with the location of origination (e.g., atria or ventricle). Thus, the term “tachycardia” is used to refer a fast heartbeat (i.e., a resting heart rate of greater than 100 beats per minute), and the term “bradycardia” is used to refer to a slow heartbeat (i.e., a resting heartbeat of less than 60 beats per minute). While many arrhythmias do not cause signs or symptoms, noticeable arrhythmia symptoms include, but are not limited to, fluttering in the chest, racing heartbeat (tachycardia), slow heartbeat (bradycardia), chest pain, shortness of breath, anxiety, fatigue, lightheadedness, diszziness, sweating and fainting.

The term “subject” or “host organism,” as used herein, refers to any individual or patient to which the subject methods are performed. Generally, the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “biological sample,” refers to any sample taken from a participant, including but not limited to cells, blood, tissue, skin, urine, etc.

The terms “administration” or “administering” refers to local and systemic administration, e.g., including enteral, parenteral, pulmonary, and topical/transdermal administration. Routes of administration for compounds (e.g., a polynucleotide encoding TNNI3) that find use in the methods described herein include, e.g., oral (per os (P.O.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device, e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intraarterial, intrarenal, intraurethral, intracardiac, intracoronary, intramyocardial, intradermal, epidural, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering a compound or composition to a mammal such that the compound or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “co-administering” or “concurrent administration”, when used, for example with respect to the compounds (e.g., TNNI3 polynucleotides) and/or analogs thereof and another active agent, refers to administration of the compound and/or analogs and the active agent such that both can simultaneously achieve a physiological effect. The two agents, however, need not be administered together. In various embodiments, administration of one agent can precede administration of the other. Simultaneous physiological effect need not necessarily require presence of both agents in the circulation at the same time. However, in certain embodiments, co-administering typically results in both agents being simultaneously present in the body (e.g., in the plasma) at a significant fraction (e.g., 20% or greater, 30% or 40% or greater, 50% or 60% or greater, 70% or 80% or 90% or greater) of their maximum serum concentration for any given dose.

The term “therapeutically effective amount” or “effective amount” means the amount and/or dosage and/or dosage regime of a compound (e.g., gene therapy vectors) or pharmaceutical composition that elicits the biological or medical response (e.g., increased expression of TNNI3) in a tissue, system, animal or human that is being sought by the researcher, veterinarian, medical doctor or other clinician. Thus, the term “therapeutically effective amount” is used herein to denote any amount of a formulation that causes a substantial improvement in a disease condition when applied to the affected areas repeatedly over a period of time. The amount varies with the condition being treated, the stage of advancement of the condition, and the type and concentration of formulation applied. Appropriate amounts in any given instance will be readily apparent to those skilled in the art or capable of determination by routine experimentation. In the context of TNNI3, an example of a therapeutically effective amount of an agent, such as a vector containing a nucleic acid encoding TNNI3 and a cardiomyocte specific promoter, is an amount sufficient to induce expression of TNNI3 in cardiomyocytes in the patient.

A “dosage” or “dose” are defined to include a specified size, frequency, or exposure level are included within the definition.

A “therapeutic effect,” as used herein, encompasses a therapeutic benefit and/or a prophylactic benefit as described herein.

As used herein, the terms “treating” and “treatment” refer to delaying the onset of, retarding or reversing the progress of, reducing the severity of, or alleviating or preventing either the disease or condition to which the term applies, or one or more symptoms of such disease or condition. The condition can include a predisposition to a disease or disorder. The effect of the administration of the composition to the subject (either treating and/or preventing) can be, but is not limited to, the cessation of one or more symptoms of the condition, a reduction or prevention of one or more symptoms of the condition, a reduction in the severity of the condition, the complete ablation of the condition, a stabilization or delay of the development or progression of a particular event or characteristic, or minimization of the chances that a particular event or characteristic will occur.

The term “mitigating” refers to reduction or elimination of one or more symptoms of that pathology or disease, and/or a reduction in the rate or delay of onset or severity of one or more symptoms of that pathology or disease, and/or the prevention of that pathology or disease. In various embodiments, the reduction or elimination of one or more symptoms of pathology or disease can include, e.g., measurable and sustained increase in the expression levels of TNNI3 in cardiomyocytes of the mammalian heart.

The term “polynucleotide” refers to a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

The terms “gene transfer” or “gene delivery” refer to methods or systems for reliably inserting foreign DNA into host cells. Such methods can result in transient expression of non-integrated transferred DNA, extrachromosomal replication and expression of transferred replicons (e.g. episomes), or integration of transferred genetic material into the genomic DNA of host cells.

As used herein, a “vector” is a tool that allows or facilitates the transfer of an entity from one environment to another. It is a plasmid, phage, or cosmid, into which another DNA segment may be inserted so as to bring about the replication of the inserted segment in the appropriate prokaryotic or eukaryotic cell. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art.

The term “viral vector” refers to a vector wherein virally-derived polynucleotide sequences are present in the vector for transfection into a host cell. Thus, viral vectors can be particularly useful for introducing a polynucleotide useful in performing a method of the invention into a target cell. Viral vectors have been developed for use in particular host systems, particularly mammalian systems and include, for example, retroviral vectors, other lentivirus vectors such as those based on the human immunodeficiency virus (HIV), adenovirus vectors (AV), adeno-associated virus vectors (AAV), herpes virus vectors, vaccinia virus vectors, and the like (see Miller and Rosman, BioTechniques 7:980-990, 1992; Anderson et al., Nature 392:25-30 Suppl., 1998; Verma and Somia, Nature 389:239-242, 1997; Wilson, New Engl. J. Med. 334:1185-1187 (1996), each of which is incorporated herein by reference). Lentivirus vectors have been most commonly used to achieve chromosomal integration.

An adeno-associated virus (AAV) is a small replication-defective, nonenveloped virus that depends on the presence of a second virus, such as adenovirus or herpes virus, for its growth in cells. Thus, the term “AAV vector” refers to a vector derived from an adeno-associated virus serotype, including without limitation, AAV1, AAV2, AAV3, AAV4, AAV5, AAV6, etc. In various embodiments, the AAV is an AAV9 particle. AAV vectors can have one or more of the AAV wild-type genes deleted in whole or part, e.g., the rep and/or cap genes, but retain functional flanking inverted terminal repeat (ITR) sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, as long as the sequences provide for functional rescue, replication and packaging. AAV expression vectors are constructed using known techniques to at least provide as operatively linked components in the direction of transcription, control elements including a transcriptional initiation region, the DNA of interest (i.e., the TNNI3 gene) and a transcriptional termination region.

Additional references describing AAV vectors which could be used in the methods of the present invention include the following: Carter, B., Handbook of Parvoviruses, vol. I, pp. 169-228, 1990; Berns, Virology, pp. 1743-1764 (Raven Press 1990); Carter, B., Curr. Opin. Biotechnol., 3: 533-539, 1992; Muzyczka, N., Current Topics in Microbiology and Immunology, 158: 92-129, 1992; Flotte, T. R., et al., Am. J. Respir. Cell Mol. Biol. 7:349-356, 1992; Chatterjee et al., Ann. NY Acad. Sci., 770: 79-90, 1995; Flotte, T. R., et al., WO 95/13365 (18 May 1995); Trempe, J. P., et al., WO 95/13392 (18 May 1995); Kotin, R., Human Gene Therapy, 5: 793-801, 1994; Flotte, T. R., et al., Gene Therapy 2:357-362, 1995; Allen, J. M., WO 96/17947 (13 Jun. 1996); and Du et al., Gene Therapy 3: 254-261, 1996. See also, U.S. Pat. No. 8,865,881, incorporated herein by reference.

If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide agent can be introduced into a cell by various methods, including, for example, by engineering the peptide to contain a protein transduction domain such as the human immunodeficiency virus TAT protein transduction domain, which can facilitate translocation of the peptide into the cell. In addition, there are a variety of biomaterial-based technologies such as nano-cages and pharmacological delivery wafers (such as used in brain cancer chemotherapeutics) which may also be modified to accommodate this technology.

As used herein, the term “gene” means the deoxyribonucleotide sequences comprising the coding region of a structural gene. A “gene” may also include non-translated sequences located adjacent to the coding region on both the 5′ and 3′ ends such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogenous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, a “regulatory gene” or “regulatory sequence” is a nucleic acid sequence that encodes products (e.g., transcription factors) that control the expression of other genes.

As used herein, a “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from eukaryotic mRNA, genomic DNA sequences from eukaryotic DNA, and synthetic nucleic acids. A transcription termination sequence will usually be located 3′ to the coding sequence.

As used herein, a “promoter” is defined as a regulatory DNA sequence generally located upstream of a gene that mediates the initiation of transcription by directing RNA polymerase to bind to DNA and initiating RNA synthesis. A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular compound or protein), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process). Thus, in various embodiments, the promoter may be a cardiac-specific promoter that drives transgene expression in cardiomyocytes of the heart. Expression vectors and plasmids in accordance with the present invention may include one or more constitutive promoters, such as viral promoters or promoters from mammalian genes that are generally active in promoting transcription. Exemplary promoters include, but are not limited to, human Elongation Factor 1 alpha promoter (EFS), SV40 early promoter, mouse mammary tumor virus long terminal repeat (LTR) promoter; adenovirus major late promoter (Ad MLP); a herpes simplex virus (HSV) promoter, an endogenous cellular promoter that is heterologous to the gene of interest, a cytomegalovirus (CMV) promoter such as the CMV immediate early promoter region (CMVIE), a Rous sarcoma virus (RSV) promoter, synthetic promoters, hybrid promoters, and the like. In various embodiments, the promoter is a tissue specific promoter, such as a promoter derived from the human TNNT2 gene (TNN2 proximal promoter) or a synthetic cardiac promoter, such as C5C12.

As used herein, an “enhancer” is a short (50-1500 bp) region of DNA that can be bound by proteins (activators) to increase the likelihood that transcription of a particular gene will occur. Thus, an enhancer may be used to increase promoter strength with regard to expression of the open reading frame for gene expression. In various embodiments, the enhancer may be fused to the cardiac-specific promoter. Exemplary enhancers useful in the gene therapy vectors described herein include, but are not limited to, the 2RS5 muscle enhancer.

As used herein, the terms “functionally linked” and “operably linked” are used interchangeably and refer to a functional relationship between two or more DNA segments, in particular gene sequences to be expressed and those sequences controlling their expression. For example, a promoter/enhancer sequence, including any combination of cis-acting transcriptional control elements is operably linked to a coding sequence if it stimulates or modulates the transcription of the coding sequence in an appropriate host cell or other expression system. Promoter regulatory sequences that are operably linked to the transcribed gene sequence are physically contiguous to the transcribed sequence.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, e.g., TNNI3 polynucleotide or polypeptide sequence as described herein, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, for example, over a region that is 50, 100, 200, 300, 400 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to TNNI3 nucleic acids and proteins, the BLAST and BLAST 2.0 algorithms and the default parameters are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., eds., Current Protocols in Molecular Biology (1995 supplement)). Examples of algorithms that are suitable for determining percent sequence identity and sequence similarity are the BLAST and BLAST 2.0 algorithms, which are described in Altschul et al., J. Mol. Biol. 215:403-410 (1990) and Altschul et al., Nucleic Acids Res. 25:3389-3402 (1977), respectively. Software for performing BLAST analyses is publicly available through the National Center for Biotechnology Information (on the worldwide web at ncbi.nlm.nih.gov/).

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “antibody” as used herein refers to polyclonal and monoclonal antibodies and fragments thereof, and immunologic binding equivalents thereof. The term “antibody” refers to a homogeneous molecular entity, or a mixture such as a polyclonal serum product made up of a plurality of different molecular entities, and broadly encompasses naturally-occurring forms of antibodies (for example, IgG, IgA, IgM, IgE) and recombinant antibodies such as single-chain antibodies, chimeric and humanized antibodies and multi-specific antibodies. The term “antibody” also refers to fragments and derivatives of all of the foregoing, and may further comprise any modified or derivatized variants thereof that retains the ability to specifically bind an epitope. Antibody derivatives may comprise a protein or chemical moiety conjugated to an antibody. A monoclonal antibody is capable of selectively binding to a target antigen or epitope. Antibodies may include, but are not limited to polyclonal antibodies, monoclonal antibodies (mAbs), humanized or chimeric antibodies, camelized antibodies, single chain antibodies (scFvs), Fab fragments, F(ab′)₂ fragments, disulfide-linked Fvs (sdFv) fragments, for example, as produced by a Fab expression library, anti-idiotypic (anti-Id) antibodies, intrabodies, nanobodies, synthetic antibodies, and epitope-binding fragments of any of the above.

As used herein, “pharmaceutically acceptable carrier” encompasses any of the standard pharmaceutical carriers, such as a phosphate buffered saline solution, water and emulsions such as an oil/water or water/oil emulsion, and various types of wetting agents.

Troponin 1.3, cardiac muscle, is a protein that in humans is encoded by the TNN13 gene. The TNNI3 gene encoding cardiac troponin I (CHM is located at 19q13.4 in the human chromosomal genome. Human cTnI is a 24 kDa protein consisting of 210 amino acids with isoelectric point (pI) of 9,87. Mutations in cTnI can result in hypertrophic, restrictive, and dilated cardiomyopathies (HCM, RCM and DCM), which culminate in thickening of the ventricular walls, cardiomyocyte disarray and thinning of the ventricular walls respectively. Ultimately, all forms result in reduced cardiac function. A hereditary heart disorder characterized by ventricular hypertrophy (HCM) is most common among TNNI3 mutations. HCM is usually asymmetric and often involves the interventricular septum. The symptoms include dyspnea, syncope, collapse, palpitations, and chest pain. They can be readily provoked by exercise. The disorder has inter- and interfamilial variability ranging from benign to malignant forms with high risk of cardiac failure and sudden cardiac death.

iPS cells derived from a patient carrying the 470 c>t mutation in the TNNI3 gene were engineered to express cardiac specific Zeocin resistance. Subsequent work was focused on engineering lentivirus and rAAV to express the wild type and mutant forms of TNNI3 for use in gene therapy. Thus, the present disclosure is based on the observation of successful viral delivery of TNNI3 controlled by a cardiac-specific promoter to cardiomyocytes in a mammalian subject. Since AAV9 has high tropism for heart tissue and cardiac specific promoters reduce toxicity associated with expression in off-target tissue, the present invention focuses on cardiomyopathies caused by TNNI3 genetic mutations.

Generally, the gene therapy vectors described herein include an expression cassette comprising a polynucleotide encoding Troponin I (TNNI3), that allows for the expression of cardiac troponin I (cTnI) to partially or wholly rectify deficient cTnI protein expression levels (e.g., haploinsuffiency, gain of function, loss of function), to correct cardiomyopathies caused by TNNI3 genetic mutations, and/or to prevent, mitigate, ameliorate, reduce or reverse one or more symptoms associated with arrhythmia in a subject in need thereof (e.g., a subject having cardiomyopathy and/or arrhythmia at least in part due to deficient TNNI3 expression). The gene therapy vectors can be viral or non-viral vectors. Illustrative non-viral vectors include, e.g., naked DNA, cationic liposome complexes, cationic polymer complexes, cationic liposome-polymer complexes, and exosomes.

Examples of viral vector include, but are not limited, to adenoviral, retroviral, lentiviral, herpesvirus and adeno-associated virus (AAV) vectors. Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins which mediate cell transduction.

Such recombinant viruses may be produced by techniques known in the art, e.g., by transfecting packaging cells or by transient transfection with helper plasmids or viruses. Typical examples of virus packaging cells include but are not limited to PA317 cells, PsiCRIP cells, GPenv+ cells, 293 cells, etc. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478, the complete contents of each of which is hereby incorporated by reference.

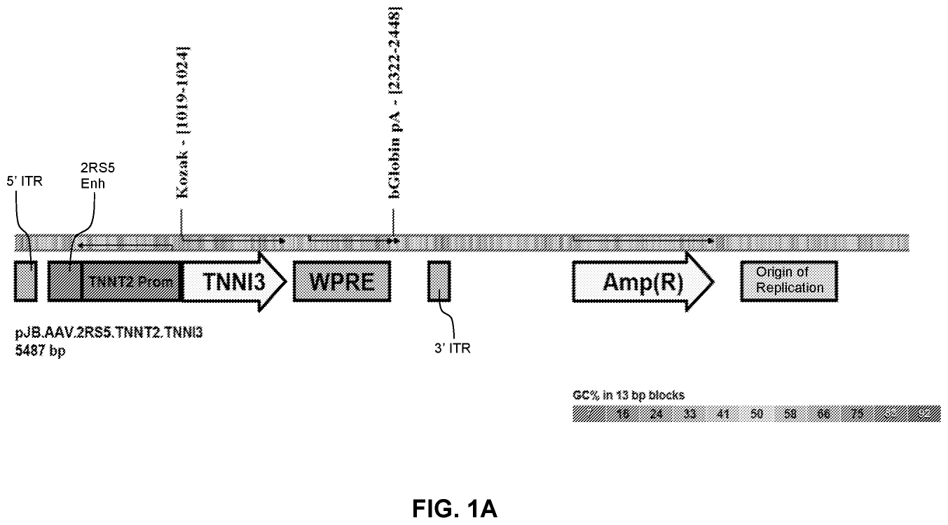

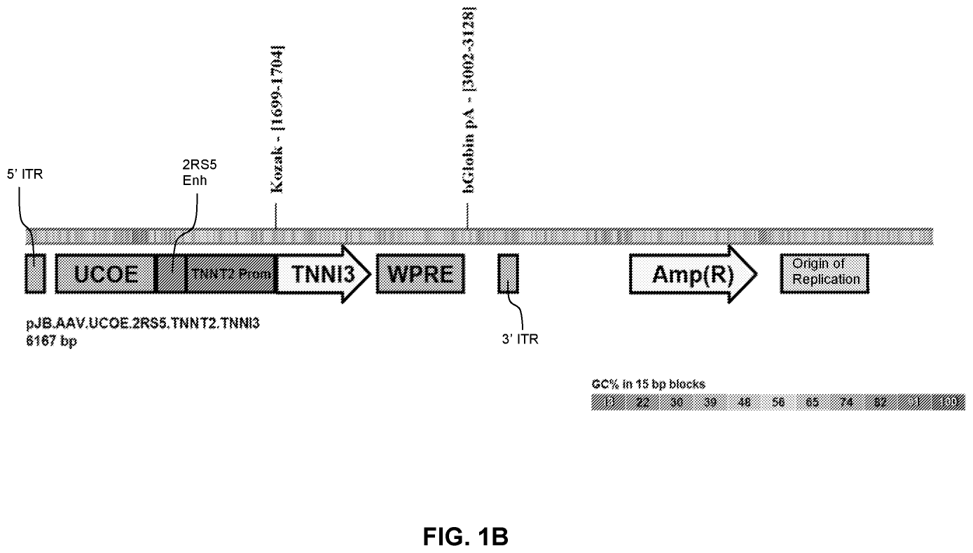

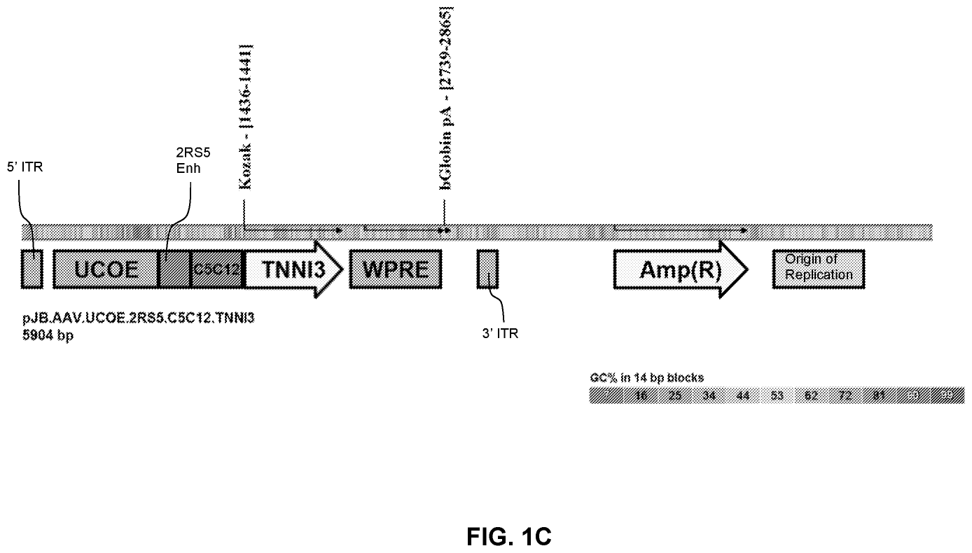

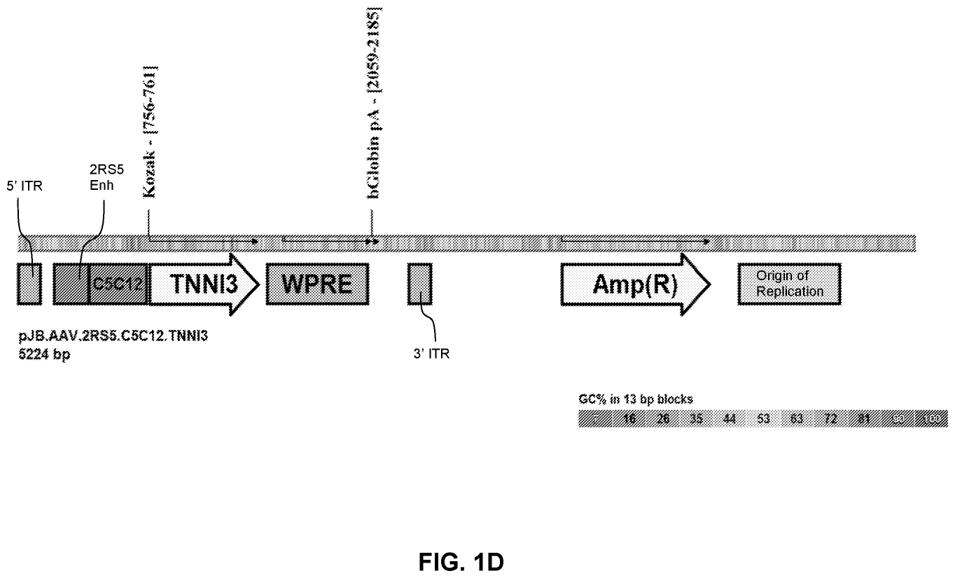

In various embodiments, the gene viral vector is an adenoviral vector or an adeno-associated viral (AAV) vector. In varying embodiments, the AAV vector is selected from AAV serotypes AAV1, AAV2, AAV3, AAV4, AAS, AAV6, AAV7, AAV8, AAV9, AAV10, AAV11, AAVrh10, and subgroups and mixtures thereof including self-complementary AAV (scAAV) genomes, or any other serotypes of AAV that can infect humans, monkeys or other species. In one embodiment, the AAV vector is an AAVrh10. In others embodiment the AAV vector is AAV9. In still another embodiment the AAV vector is AAV8. Recombinant AAV (rAAV) vectors are frequently utilized for the delivery of therapeutic genes and have been studied in human clinical trials. rAAV vectors can be designed to deliver specified transgenes to a patient's cells for expression. After infection and introduction of the viral genome by the rAAV vector, the viral genes exist mostly as extrachromosomal structures that do not integrate into the host's genome, but are expressed by the host cell's translational machinery. For successful host cell infection and gene expression, rAAV vectors require several components (see, e.g., FIGS. 1A-1D): inverse terminal repeat elements (ITRs), promoter and/or enhancer region, transgene, and 3′ untranslated region. After viral infection of host cells, the promoter region initiates signals for translation of the virally delivered transgene by the host cell's translational machinery.

Generally, the control elements are selected to be functional in a mammalian cell. The resulting construct which contains the operatively linked components is bounded (5′ and 3) with functional AAV ITR sequences. By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, e.g., Kotin, 1994; Berns, K I “Parvoviridae and their Replication” in Fundamental Virology, 2nd Edition, (B. N. Fields and D. M. Knipe, eds., the complete contents of which is hereby incorporated by reference) for the AAV2 sequence. As used herein, an “AAV ITR” does not necessarily include the wild-type nucleotide sequence, but may be altered, e.g., by the insertion, deletion or substitution of nucleotides.

Additionally, the AAV ITR may be derived from any of several AAV serotypes, including without limitation, AAV1, AAV2, AAV3, AAV4, AAS, AAV6, AAV7, AAV8, AAV9, AAV10, AAV11, AAVrh10. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell.

In varying embodiments, vectors derived from AAV serotypes having tropism for, and high transduction efficiencies in, cells of the mammalian myocardium are employed, particularly cardiomyocytes and cardiomyocyte progenitors. A review and comparison of transduction efficiencies of different serotypes is provided in Cearley, et al., Molecular Therapy 16(10); 1710-1718, 2008, the complete contents of which is hereby incorporated by reference. In other non-limiting examples, preferred vectors include vectors derived from any serotypes like AAV1, AAV2, AAV3, AAV4, AAS, AAV6, AAV7, AAV8, AAV9, AAV10, AAV11 or AAVrh10, which have also been shown to transduce cells of cardiomyocytes.

In various embodiments, the selected nucleotide sequence is operably linked to control elements that direct the transcription or expression thereof in the subject in vivo. Such control elements can include control sequences normally associated with the selected gene.

Alternatively, heterologous control sequences can be employed. Useful heterologous control sequences generally include those derived from sequences encoding mammalian or viral genes. Examples include, but are not limited to, the phophoglycerate kinase (PGK) promoter, CAG (CMV enhancer with chicken beta-actin promoter including sequence through the 1st intron and the splice acceptor of the rabbit beta-globin gene) promoter, MCK (muscle creatine kinase) promoter, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), rous sarcoma virus (RSV) promoter, synthetic promoters, hybrid promoters, and the like. The promoters can be of human origin or from other species, including from mice. In addition, sequences derived from non-viral genes, such as the marine metallothionein gene, will also find use herein. Such promoter sequences are commercially available from, e.g. Stratagene (San Diego, Calif.). Examples of heterologous promoters include but are not limited to the CMV promoter. Examples of inducible promoters include but are not limited to DNA responsive elements for ecdysone, tetracycline, and hypoxia andaufin.

Similarly, control elements at the 3′ end of a coding region, including a 3′ untranslated region (3′ UTR) and/or a polyadenylation signal, can derive from the inserted gene or from a heterologous source. In various embodiments, the gene therapy vectors provided herein utilize the woodchuck hepatitis virus posttranscriptional regulatory element (WPRE) as a 3′ UTR or a rabbit beta-globin polyadenylation signal or both (see, e.g., FIGS. 1A-1D). 3′ control elements can also include control elements connecting to coding regions.

The AAV expression vector which harbors the DNA molecule of interest bounded by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publications Nos. WO 92/01070 (published 23 Jan. 1992) and WO 93/03769 (published 4 Mar. 1993); Lebkowski et al., 1988; Vincent et al., 1990; Carter, 1992; Muzyczka, 1992; Kotin, 1994; Shelling and Smith, 1994; and Zhou et al., 1994, the complete contents of each of which is hereby incorporated by reference.

Alternatively, AAV ITRs can be excised from the viral genome or from an AAV vector containing the same and fused 5′ and 3′ of a selected nucleic acid construct that is present in another vector using standard ligation techniques. AAV vectors which contain ITRs have been described in, e.g., U.S. Pat. No. 5,139,941, the complete contents of which is hereby incorporated by reference. In particular, several AAV vectors are described therein which are available from the American Type Culture Collection (“ATCC”) under Accession Numbers 53222, 53223, 53224, 53225 and 53226. Additionally, chimeric genes can be produced synthetically to include AAV ITR sequences arranged 5′ and 3′ of one or more selected nucleic acid sequences. Preferred codons for expression of the chimeric gene sequence in mammalian CNS cells can be used. The complete chimeric sequence is assembled from overlapping oligonucleotides prepared by standard methods. See, e.g., Edge Nature, vol, 292, 1981, page 756; Nambair et al., Science, vol. 223, 1984, page 1299; Jay et al., J. Biol. Chem. vol. 259, 1984, page 6311, the complete contents of each of which is hereby incorporated by reference. In order to produce AAV virions, an AAV expression vector is introduced into a suitable host cell using known techniques, such as by transfection. A number of transfection techniques are generally known in the art. See, e.g., Graham et al, Virology, 52, 456-467, (1973); Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Particularly suitable transfection methods include calcium phosphate co-precipitation (Graham et al., 1973), direct microinjection into cultured cells (Capeechi, 1980), electroporation (Shigekawa et al., 1988), liposome mediated gene transfer (Mannino et al., 1988), lipid-mediated transduction (Feigner et al., 1987, PNAS USA, 84, 21, 7413-17), and nucleic acid delivery using high-velocity microprojectiles (Klein et al., 1987, Endocrinology 120:2339-45). The complete contents of each of the foregoing references are hereby incorporated by reference in entirety.

In varying embodiments structure of the expression cassette within the vector comprises first and second (e.g., 5′ and 3′) inverse terminal repeats (ITRs) from any known AAV serotype or subgroup including scAAV, any known promoter region (e.g., a cardiac promoter) with or without any known enhancer element (e.g., a muscle enhancer), the TNNI3 gene, and a posttranscriptional regulatory element (e.g., WPRE). In various embodiments, expression cassette includes an enhancer fused to a cardiac promoter such that the resultant promoter system is one of various arrangements which are not permissive to TNNI3 gene expression outside of the heart. An appropriate serotype such as AAV9 and AAV8 promote more specific transduction of cardiomyocytes in heart tissue.

FIG. 1A shows an exemplary AAV vector coding for the 2RS5 muscle enhancer fused to the TNNT2 proximal promoter. FIG. 1B shows an exemplary AAV vector coding for a ubiquitous chromatin opening element (UCOE) derived from the CBX3 gene promoter. The UCOE provides approximately 3500 bp of protection from methylation-based gene silencing in the direction of the TNNI3 coding sequence. In various embodiments, the UCOE is positioned upstream of the 2RS5 muscle enhancer fused to the TNNT2 proximal promoter. FIG. 1C shows an exemplary AAV vector coding for a UCOE positioned upstream of the 2RS5 muscle enhancer fused to a synthetic cardiac promoter C5C12. FIG. 1D shows an exemplary AAV vector coding for the 2RS5 muscle enhancer fused to a synthetic cardiac promoter (C5C12).

Translation of the virally delivered TNNI3 gene results in expression of the cTnI protein, which is then targeted to cardiomyocites of the heart. Without being bound by theory, the most common non-truncating TNNI3 mutations result in aberrant function of the thin filament within the sarcomere of the cardiac myofiber. Those mutations that result in truncation of the TNNI3 gene (frameshift mutations) typically result in a haploinsufficient condition where low titers of cTnI protein cause aberrant thin filament activity. Restoration of TNNI3 expression then restores or ameliorates myofiber thin filament function, which is deficient and causal in cardiomyopathy and/or arrhythmia. Ultimately, restoration of TNNI3 expression serves to treat the underlying genetic deficiency that causes cardiomyopathy and relieves the phenotype and symptoms of the disease. In other disorders where TNNI3 may be expressed, but not at a sufficient level (such as in the case of haploinsufficiency), delivery of the transgenic TNNI3 by the rAAV vector results in overexpression of the resultant cTnI protein, thus restoring proper thin filament function.

In another aspect, the invention provides pharmaceutical compositions for use in preventing or treating cardiomyopathy and/or one or more symptoms associated with arrhythmia at least in part due to deficient TNNI3 expression, where the compositions include a therapeutically effective amount of a vector that includes a nucleic acid sequence encoding TNNI3.

It will be understood that the single dosage or the total daily dosage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific nucleic acid or polypeptide employed; and like factors well known in the medical arts. For example, it is well within the skill of the art to start doses of a compound at levels lower than those required to achieve the desired therapeutic effect and to gradually increase the dosage until the desired effect is achieved. However, the daily dosage of the products may be varied over a wide range per adult per day. The therapeutically effective amount of the vector according to the invention that should be administered, as well as the dosage for the treatment of a pathological condition with the number of viral or non-viral particles and/or pharmaceutical compositions described herein, will depend on numerous factors, including the age and condition of the patient, the severity of the disturbance or disorder, the method and frequency of administration and the particular peptide to be used.

The pharmaceutical compositions that contain the vector may be in any form that is suitable for the selected mode of administration, for example, for intraventricular, intramyocardarial, intracoronary, intravenous, intra-arterial, intra-renal, intraurethral, epidural or intramuscular administration. The gene therapy vector comprising a polynucleotide encoding TNNI3 can be administered, as the sole active agent, or in combination with other active agents, in a unit administration form, as a mixture with conventional pharmaceutical supports, to animals and human beings.

In various embodiments, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and storage and must be preserved against the contaminating action of microorganisms, such as bacteria and fungi.

Solutions comprising the gene therapy vectors as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

In various embodiments, the gene therapy vectors can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also serve as a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Also provided are methods of preventing, mitigating, ameliorating, reducing, inhibiting, eliminating and/or reversing one or more symptoms of cardiomyopathy, heart failure, and/or arrhythmia in a subject in need thereof. The methods include administering to the subject a gene therapy vector as described above and herein, e.g., an adeno-associated virus (AAV) vector that includes an expression cassette comprising a polynucleotide encoding TNNI3. The vector is delivered to the subject in need thereof and whereby the polynucleotide encoding TNNI3 is expressed by the transduced cells at a therapeutically effective level. In various embodiments, the vector is an AAV9 or AAV8.

In various embodiments, the vector is administered via a route selected from the group consisting of intravenous, intraarterial, intracardiac, intracoronary, intramyocardial, intrarenal, intra-urethral, epidural, subcutaneous, and intramuscular. In varying embodiments, the vector is delivered directly into the myocardium by epicardiac injection via a minithoracotomy, by intracoronary injection, by endomyocardic injection or by another type of injection useful in the heart. Additional routes of administration may also include local application of the vector under direct visualization, e.g., superficial cortical application, or other nonstereotactic application.

Viral vectors will typically provoke an immune response which can include an antibody response that can reduce or completely inhibit the effectiveness of a particular vector in that individual when repeat dosing becomes necessary or desirable. Repeat dosing may become appropriate, for example, because vector can be lost over time due to cell proliferation, especially episomal vectors. Tissues that are more difficult to access can require multiple administrations of vector to transfect a sufficient number of cells for effective treatment of the disease. Expression of the transgene can also be lost over time. The need to administer a gene therapy vector in the face of an inhibiting antibody response can be addressed in various ways. For example, antibody titers can be reduced by apheresis prior to administration of the vector or the patient can be immunosuppressed with an appropriate medical regimen. Alternatively, empty AAV capsid could be administered to bind host antibodies and immune cells prior to injection of therapeutic AAV containing the TNNI3 transgene. Depending on the target tissue, a direct local administration instead of a systemic administration can be useful in overcoming or ameliorating the inhibitory effect of anti-vector antibodies. In yet another approach, a vector based on a different serotype of the virus from which it was derived can be used. For example, if an AAV9 vector was used initially and there becomes a problem with anti-AAV antibody titer, but there is need for further gene therapy, one can administer an AAV8 vector carrying the same (or different) gene construct.

Upon formulation, solutions can be administered in a manner compatible with the dosage formulation and in such amount as is therapeutically effective. The formulations are easily administered in a variety of dosage forms, such as the type of injectable solutions described above, but drug release capsules and the like can also be employed. Multiple doses can also be administered.

As appropriate, the vectors described herein may be formulated in any suitable vehicle for delivery. For instance, they may be placed into a pharmaceutically acceptable suspension, solution or emulsion. Suitable mediums include saline and liposomal preparations. More specifically, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but are not limited to water, alcoholic/aqueous solutions, emulsions or suspensions, including saline and buffered media. Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes.

An appropriate regimen can be determined by a physician, and will depend on the age, sex, weight, of the subject, and the stage of the disease. As an example, for delivery of a nucleic acid sequence encoding a TNNI3 polypeptide using a viral expression vector, each unit dosage of TNNI3 polypeptide expressing vector may include about 2.5 μl to 100 μl of a composition including a viral expression vector in a pharmaceutically acceptable fluid at a concentration ranging from 10¹¹ to 10¹⁶ viral genome per ml.

Also provided are kits including a gene therapy vector comprising a polynucleotide encoding TNNI3, as described above and herein. In various embodiments, the kits provide the gene therapy vectors prepared in one or more unitary dosage forms ready for administration to a subject, for example in a preloaded syringe or in an ampoule. In varying embodiments, the gene therapy vector is provided in a lyophilized form.

Amino acid sequences for human TNNI3 and TNNT2 are known in the art. See, for example, GenBank Accession No.: X90780.1, human Troponin I, cardiac muscle, which provides the amino acid sequence (SEQ ID NO: 1):

MADGSSDAAREPRPAPAPIRRRSSNYRAYATEPHAKKKSKISASRKLQL
KTLLLQIAKQELEREAEERRGEKGRALSTRCQPLELAGLGFAELQDLCR
QLHARVDKVDEERYDIEAKVTKNITEIADLTQKIFDLRGKFKRPTLRRV
RISADAMMQALLGARAKESLDLRAHLKQVKKEDTEKENREVGDWRKNID
ALSGMEGRKKKFES

Accession No. NM_000363.5, Homo sapiens troponin 12, cardiac type (TNNI3), mRNA, which provides the nucleic acid sequence (SEQ ID NO: 2):

agtgtcctcg gggagtctca agcagcccgg aggagactga
cggtccctgg gaccctgaag gtcacccggg cggccccctc
actgaccctc caaacgcccc tgtcctcgcc ctgcctcctg
ccattcccgg cctgagtctc agcatggcgg atgggagcag
cgatgcggct agggaacctc gccctgcacc agccccaatc
agacgccgct cctccaacta ccgcgcttat gccacggagc
cgcacgccaa gaaaaaatct aagatctccg cctcgagaaa
attgcagctg aagactctgc tgctgcagat tgcaaagcaa
gagctggagc gagaggcgga ggagcggcgc ggagagaagg
ggcgcgctct gagcacccgc tgccagccgc tggagttggc
cgggctgggc ttcgcggagc tgcaggactt gtgccgacag
ctccacgccc gtgtggacaa ggtggatgaa gagagatacg
acatagaggc aaaagtcacc aagaacatca cggagattgc
agatctgact cagaagatct ttgaccttcg aggcaagttt
aagcggccca ccctgcggag agtgaggatc tctgcagatg
ccatgatgca ggcgctgctg ggggcccggg ctaaggagtc
cctggacctg cgggcccacc tcaagcaggt gaagaaggag
gacaccgaga aggaaaaccg ggaggtggga gactggcgca
agaacatcga tgcactgagt ggaatggagg gccgcaagaa
aaagtttgag agctgagcct tcctgcctac tgcccctgcc
ctgaggaggg ccctgaggaa taaagcttct ctctgagctg
aaa

The following examples are intended to illustrate but not limit the invention.

### Example 1

### AAV9 is Infectious in iPSC Derived Cardiomyocytes

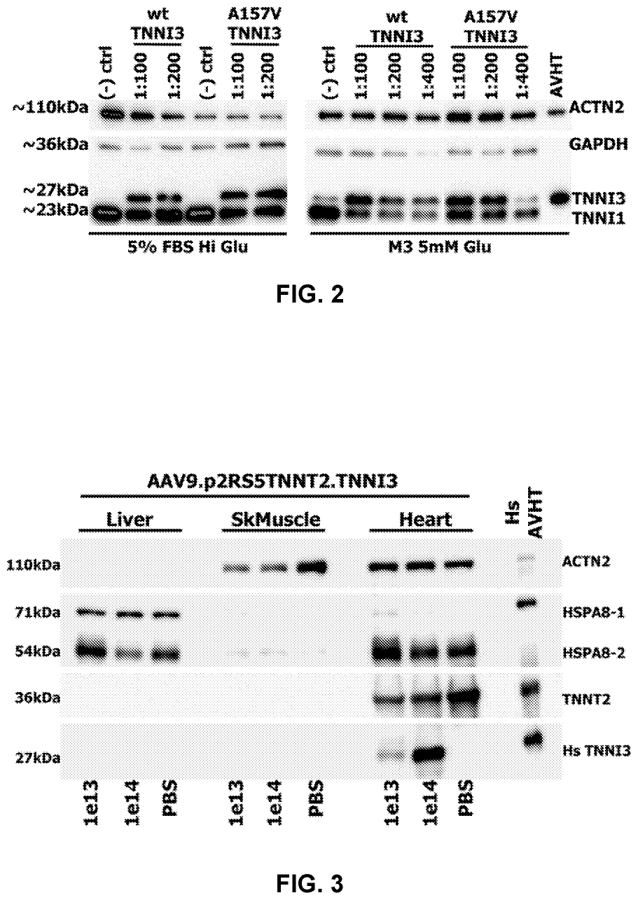

Particular vectors, consistent with the descriptions above, were constructed as AAV9 vectors. As shown in FIGS. 1A and 2, clonal iPS cell-derived fetal cardiomyocytes were transduced with rAAV9-2RS5TNNT2-TNNI3 to demonstrate delivery of ectopic TNNI3 to cells that express no detectable cTnI (see 5% FBS media in FIG. 2) or that express increasing levels of cTnI when grown in maturation media (see M3 media in FIG. 2). In this example, iPSC derived cardiomyocytes were infected with a multiplicity of infection of between 2 and 4 thousand viral genomes. As discussed above, the AAV9 serotype is tropic for liver, heart and skeletal muscle. In this example, efficient production of TNNI3 transcripts from the cardiac TNNT2 promoter is visualized as ectopic cTnI protein in fetal cardiomyocytes. As a reference, adult ventricular heart tissue lysate is run alongside test samples (FIG. 2, AVHT). As a comparison, AVHT cTnI illustrates both the maturity and the abundance of the slow skeletal TnI (ssTnI) and cTnI isoforms as the antibody recognizes both proteins equally.

### Example 2

### Administration of AAV9 TNNI3 Vectors in a Mouse Model

An animal was developed to validate the drug for the rare TNNI3 non-truncating variant 470 c>t (A157V). Wild type mice were injected with PBS, 1e+13, or 1e+14 vg of AAV9.TNNT2.TNNI3. At four weeks post injection, the level and specificity of TNNI3 gene expression was evaluated in the Liver, Skeletal Muscle (SkMuscle), and Heart tissue (FIG. 3). Specific expression was dose dependent in only heart tissue as indicated. Endogenous TNNT2 protein expression indicates cardiomyocyte specific tissue, whereas ACTN2 indicates expression in only myocytes. HSPA8-1/2 was blotted as a housekeeping gene control. Note that typically the liver absorbs about 10-fold more virus than the heart, yet there is no detectable expression of cTnI. This demonstrates that the expression of ectopic TNNI3 transcripts and expression of human cTnI is restricted to the heart tissue. Note that the antibody used in the western blot in FIG. 3 does not recognize ssTnI and is specific to human cTnI.

In summary, these examples show that gene therapy vectors based on adeno-associated virus encoding TNNI3 can be administered intravenously to successfully achieve transgene expression in heart tissue. Additionally, such expression leads to the reversal of one or more symptoms associated with cardiomyopathy, heart failure, and/or arrhythmia. These data support the use of such vectors for gene therapy in the treatment of cardiomyopathy, heart failure, and arrhythmia.

Although the invention has been described with reference to the above examples, it will be understood that modifications and variations are encompassed within the spirit and scope of the invention. Accordingly, the invention is limited only by the following claims.

1. A gene therapy vector comprising an expression cassette comprising a polynucleotide encoding a cardiac-specific promoter operably linked to a polynucleotide encoding a functional human TNNI3 gene.

2. The gene therapy vector of claim 1, wherein the vector is a viral vector selected from the group consisting of adenovirus, retrovirus, lentivirus, herpesvirus and adeno-associated virus (AAV).

3. The gene therapy vector of claim 2, wherein the vector is from one or more of adeno-associated virus (AAV) serotypes 1-11, or any subgroups thereof.

4. The gene therapy vector of claim 3, wherein the viral vector is encapsulated in an anionic liposome.

5. The gene therapy vector of claim 1, wherein the vector is a non-viral vector selected from the group consisting of naked DNA, a cationic liposome complex, a cationic polymer complex, a cationic liposome-polymer complex, and an exosome.

6. The gene therapy vector of claim 1, wherein the expression cassette comprises operably linked in the 5′ to 3′ direction, a first inverse terminal repeat, an enhancer fused to the cardiac-specific promoter, the polynucleotide encoding TNNI3, a posttranscriptional regulatory element, and a second inverse terminal repeat.

7. The gene therapy vector of claim 6, wherein the expression cassette further comprises a ubiquitous chromatin open element positioned upstream of the enhancer.

8. The gene therapy vector of claim 6, wherein the cardiac-specific promoter is selected from the group consisting of TNNT2 proximal promoter and C5C12 synthetic promoter.

9. The gene therapy vector of claim 6, wherein the enhancer is a 2RS5 muscle enhancer.

10. The gene therapy vector of claim 1, wherein the polynucleotide comprises DNA or cDNA.

11. The gene therapy vector of claim 1, wherein the polynucleotide encoding TNNI3 has at least about 90% sequence identity to SEQ ID NO: 2.

12. (canceled)

13. A method of preventing, mitigating, ameliorating, reducing, inhibiting, eliminating and/or reversing one or more symptoms of cardiomyopathy or arrhythmia in a subject in need thereof, comprising administering to the subject the gene therapy vector of claim 1.

14. A method of preventing, mitigating, ameliorating, reducing, inhibiting, eliminating and/or reversing one or more symptoms of cardiomyopathy or arrhythmia in a subject in need thereof, comprising administering to the subject an adeno-associated virus (AAV) vector comprising an expression cassette comprising a polynucleotide encoding TNNI3.

15. The method of claim 14, wherein the expression cassette further comprises a cardiac-specific promoter operably linked to the polynucleotide encoding TNNI3.

16. The method of claim 15, wherein the expression cassette comprises operably linked in the 5′ to 3′ direction, a first inverse terminal repeat, an enhancer fused to the cardiac-specific promoter, the polynucleotide encoding TNNI3, a posttranscriptional regulatory element, and a second inverse terminal repeat.

17. The method of claim 16, wherein the expression cassette further comprises a ubiquitous chromatin open element positioned upstream of the enhancer.

18. (canceled)

19. (canceled)

20. The method of claim 14, wherein the vector is administered via a route selected from the group consisting of intravenous, intra-arterial, intracardiac, intracoronary, intramyocardial, intrarenal, intraurethral, epidural, and intramuscular.

21. The method of claim 14, wherein the vector is administered multiple times.

22. (canceled)

23. The method of claim 14, wherein the subject has been identified as having a mutated TNNI3 gene.

24. The method of claim 14, wherein the subject has been identified as having progressive cardiomyopathy or end-stage heart failure.

25. (canceled)
